(19) United States
(12) Patent Application Publication
(10) Pub. No.: US 2006/0243287 A1
(43) Pub. Date: Nov. 2, 2006

(54) A Surgical Method for Implanting Anchored Intervertebral Disc Space Devic

(76) Inventors: Merrill Wayne Reuter, Boca Raton, FL (US); Gerald Louis Reuter, Highland Beach, FL (US)

Correspondence Address: GERALD L. REUTER, 3101 S. OCEAN BLVD., # 518, HIGHLAND BEACH, FL, 33487, US

(21) Appl. No.: 10/908,211

(22) Filed: May 2, 2005

(51) Int. Cl. A61B 19/00

(52) U.S. Cl. 128/898

(57) ABSTRACT

A method for anchoring intervertebral disc devices and or augmentation materials for treating human intervertebral disc abnormalities by minimally invasive surgical procedure. An access to the intervertebral disc space is made through the superior or inferior vertebral endplate. This endplate perforation then serves to stabilize the expandable or inflatable device or implant at the entry site of the disc space. The anchoring limits movement within the disc space as it is essentially anchored at the bony channel of the endplate opening or by direct fixation to the bony endplate. This approach is usable for permanently placed devices, and also for removable implants.

### BACKGROUND OF THE INVENTION

Intervertebral disc herniations and other abnormalities of the disc space are a major source of back pain. Herniations and injuries of intervertebral discs may cause pain and numbness in the legs, feet and arms of affected patients. Herniated or injured discs may be caused by traumatic injury due to accident, illness, the aging process as well as other undefined causes.

Intervertebral discs are located between adjacent vertebrae of the spine and comprise an annulus; a flexible wall connecting to the adjacent vertebrae, nucleus pulposus; a pulp like composition that is contained by the annulus and adjacent vertebral end plates. The nucleus pulposes provides a cushioning property at each intervertebral disc. A herniation of an intervertebral disc results from a weakened or stretched area of the annulus. The stretched area may impinge upon adjacent spinal nerves causing the pain and numbness to the specific area that the affected nerve controls.

Removing a quantity of nucleus pulposes reduces pressure in the disc space thereby relieving the pressure of the bulging annulus that is pressing on the nerve. The relief of this pressure on the nerve relieves the problem pain and numbness to the patient. Intervertebral disc distraction devices and implants enhance the disc space thereby relieving the pressure of the bulging annulus pressing against a nerve.

Intervetebral disc space devices that are not anchored to bone may move within the disc space and possibly dislodge from the space. Such issues have limited the development of devices that mimic the normal function of the nucleus pulposus and annulus fibrosis disc complex. Examples of distraction and implant devices include Furnstrum balls, Ray PDN, silicon block, rubber plates, etc. Some of these examples attempt to simulate the shock absorber effect within the disc space after disc injury, but material factors and fixation are frequent problem conditions. The instant invention provides an approach to address fixation or anchoring of flexible and non-rigid intervetrebral disc space devices and implants.

Intervertebral disc injuries and abnormalities may be treated with implantable disc spacers. An inflatable balloon distraction device as described in U.S. Pat. No. 6,805,715 is not securely anchored within the disc space. Other methods and devices as described in U.S. Pat. No. 6,146,420 require vertebral end plate preparation to hold the distraction device. The latter procedure requires open surgical procedure. U.S. Pat. No. 6,436,139 is one example of a distraction device with a designed barb to better secure the device to the vertebral end plates. Additional patents with methods and device designs intended to reduce movement within the disc space are given in References.

The surgical method of the prior art generally involves the more invasive open surgery. Open surgical procedure requires relatively long recovery times for the patient.

The open surgical procedure provides an access area that is large enough so that a microscope or other computer image enhancement technique is not required for placing the intervertebral disc space device. Magnifying eyeglasses or jewelers type magnifying loupes may be employed.

The instant invention relates to a method for treating intervertebral disc injury employing minimally invasive surgical procedure. Minimally invasive surgical procedure allows for short-term recovery from surgery and the patient's early return to normal activity.

Minimally invasive technique for purposes of the instant invention employs what may be described as a long hypodermic needle or guide for the surgical procedure with an outer diameter range from 1 mm to 9 mm. The preferred diameter for the instant invention ranges from 3.5 mm to 5 mm. This diameter range accommodates fiber inserts for light, viewing, and instrumentation. The instrumentation includes drills to create access tunnel through vertebral bone, flush tubes to remove bone particulates and flushable nucleus pulposus matter. Also, the lumen of the needle or guide tube also accommodates the transfer of the disc space distraction devices.

### REFERENCES

U.S. Pat. No. 4,772,287 Inventors; Ray et al

U.S. Pat. No. 4,898,161 Inventors; Grundei

U.S. Pat. No. 5,015,247 Inventors; Michelson

U.S. Pat. No. 5,484,437 Inventors; Michelson

U.S. Pat. No. 5,797,909 Inventors; Michelson

U.S. Pat. No. 6,080,155 Inventors; Michelson

U.S. Pat. No. 6,814,737 Inventors; Cauthen

U.S. Pat. No. 5,827,328 Inventors; Buttermann

U.S. Pat. No. 6,783,546 Inventors; Zucherman et al

U.S. Pat. No. 6,733,496 Inventors; Sharkey et al

U.S. Pat. No. 6,730,126 Inventors; Boehm

U.S. Pat. No. 6,666,891 Inventors; Boehm

U.S. Pat. No. 6,641,614 Inventors; Wagner et al

U.S. Pat. No. 6,080,193 Inventors; Hochchuler et al

U.S. Pat. No. 6,045,579 Inventors; Hochshuler et al

U.S. Pat. No. 5,827,328 Inventors; Buttermann

### BRIEF SUMMARY OF THE INVENTION

A method for treating human intervertebral disc herniations and other abnormalities of the spine using endoscopic procedure that is more particularly described as minimally invasive surgery. An access port may be made by one or more of the trajectories as shown on the drawing, FIG. 9.

A method for placing intervertebral disc devices and or augmentation devices for treating human intervertebral disc abnormalities. An access to the intervertebral disc space is made through the superior or inferior vertebral endplate. This endplate perforation then serves to stabilize the expandable, inflatable device or implant at its entry site into the disc space. This stabilized or anchored device provides limited movement within the disc space. The device is essentially anchored at the bony channel of the endplate opening or by direct fixation to the bony endplate. The approach allows for a firm disc space entry fixation that the annulus alone is incapable of providing. This approach is usable for permanently placed devices, and also for removable implants.

### BRIEF DESCRIPTION OF THE SEVERAL VIEWS OF THE DRAWINGS

FIG. 1 is a lateral view of the posterior, trans pedicular, trans superior endplate trajectory.

FIG. 2 is a top cross sectional view of the posterior, superior endplate with trans pedicular, trans superior endplate trajectory.

FIG. 3 is a lateral view of the posterior, trans pedicular, trans inferior endplate trajectory.

FIG. 4 is a bottom cross sectional view of the posterior, inferior endplate with trans pedicular, trans inferior endplate trajectory.

FIG. 5 is a lateral view of the anterior, trans superior endplate trajectory.

FIG. 6 is a top cross sectional view of the superior endplate with an anterior, trans superior endplate trajectory.

FIG. 7 is a lateral view of the anterior, trans inferior endplate trajectory.

FIG. 8 is a bottom cross sectional view of the inferior endplate with an anterior, trans inferior endplate trajectory.

FIG. 9 is a drawing of all the trajectories for singular display.

### DESCRIPTION

The present invention relates to a surgical method for treating intervertebral disc injury using endoscopic and/or fluoroscopic procedure. The method provides minimally invasive procedure that allows for short-term recovery from surgery and a patient's early return to activity.

FIG. 1 shows a lateral sectional view of a spinal motion segment with intervertebral discs C, and vertebral bodies B. The procedure involves preparing the skin for a surgical procedure, inserting an suitably sized needle type guide along a planned trajectory until bone is encountered. Then drilling though bone, either of the vertebral body B anteriorly, or the pedicle A posteriorly. Once defined by a guide wire, the trajectory can be drilled and the bony tunnel then acts as a stabilized entry site into the disc space C.

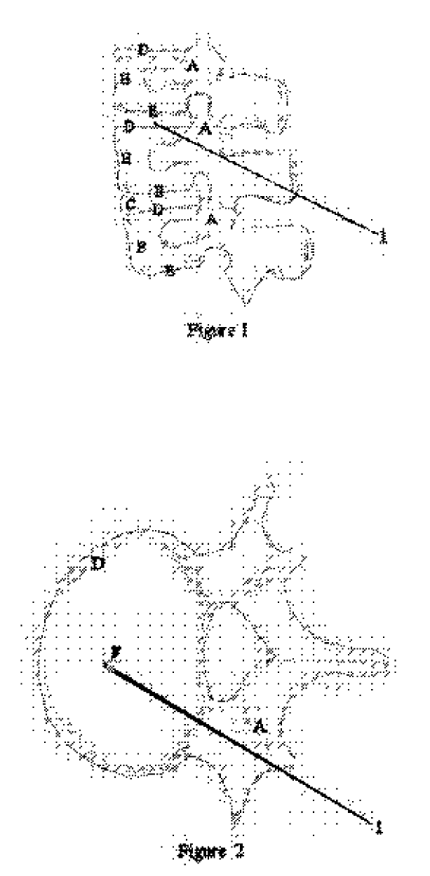

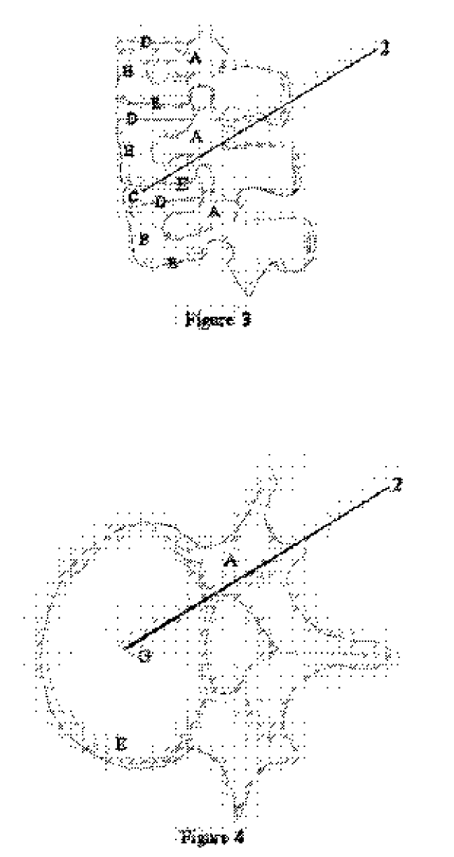

The posterior approach to the superior endplate D passes through the pedicle A as depicted in trajectory 1 and seen in FIGS. 2 and 3. FIG. 2 is the lateral view whereas FIG. 3 shows the top down view of the superior endplate with trajectory 1 coming through the bone at F.

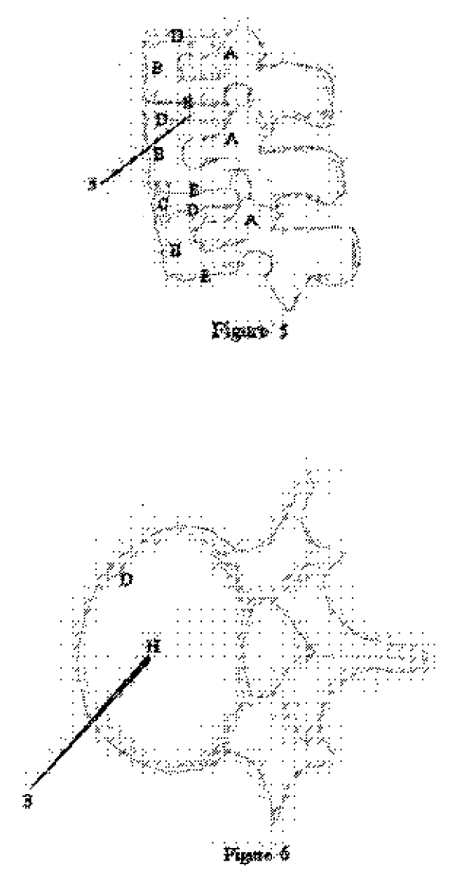

The posterior approach to the inferior endplate E passes through the pedicle A as depicted in trajectory 2 and seen in FIGS. 4 and 5. FIG. 4 is the lateral view whereas FIG. 5 shows the bottom up view of the inferior endplate with trajectory 2 coming through the bone at G.

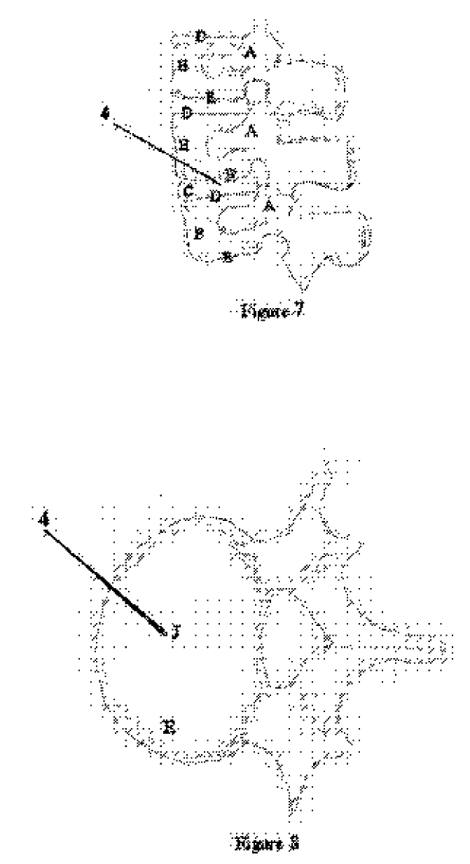

The anterior approach to the superior endplate D passes through the vertebral body B as depicted in trajectory 3 and seen in FIGS. 6 and 7. FIG. 6 is the lateral view whereas FIG. 7 shows the top down view of the superior endplate with trajectory 3 coming through the bone at H.

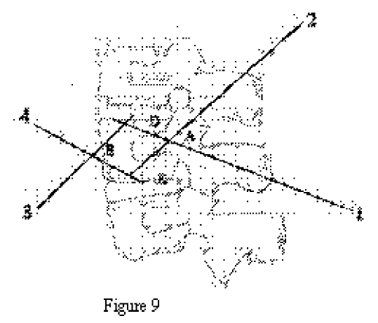

The anterior approach to the inferior endplate E passes through the vertebral body B as depicted in trajectory 4 and seen in FIGS. 8 and 9. FIG. 8 is the lateral view whereas FIG. 9 shows the bottom up view of the inferior endplate with trajectory 4 coming through the bone at J.

It is within the purview of this invention to utilize expandable or inflatable assembly constructed of a dissolvable material composition. The utilization of a dissolvable structure, which preferably would dissolve in a specified time period, would alleviate the need of the subsequent removal of the temporary assembly from the patient.

Prior to the placement of a device, a medically suitable option such as powdered hydroxyapatite or cell culture material or the like to facilitate the ingrowth of structured tissue in the intervertebral space formerly occupied by the removed pulp may be used. Homologous tissue cell culture seeding may also be used to facilitate the ingrowth of structured tissue in the operated disc space.

The procedure of the invention employs a minimally invasive procedure, which provides for a reduced cost, less time involved surgical procedure, and a patient's short-term surgical recovery and early return to activity. The procedure can be performed at all areas of the spine, including cervical, thoracic and lumbar areas.

What is claimed is:

1. A method for treating intervertebral disc abnormality, utilizing endoscopic procedure, by creating a channel through a spinal vertebra to provide an opening into the intervertebral disc space to anchor a distraction device or material for implantation.

2. The method of claim 1 wherein a posterior, trans pedicular, trans superior endplate trajectory is accomplished for treating intervertebral disc abnormality.

3. The method of claim 1 wherein a posterior, superior endplate with trans pedicular, trans superior endplate trajectory is accomplished for treating intervertebral disc abnormality.

4. The method of claim 1 wherein a posterior, trans pedicular, trans inferior endplate trajectory is accomplished for treating intervertebral disc abnormality.

5. The method of claim 1 wherein a posterior, inferior endplate with trans pedicular, trans inferior endplate trajectory is accomplished for treating intervertebral disc abnormality.

6. The method of claim 1 wherein an anterior, trans superior endplate trajectory is accomplished for treating intervertebral disc abnormality.

7. The method of claim 1 wherein a superior endplate with an anterior, trans superior endplate trajectory is accomplished for treating intervertebral disc abnormality.

8. The method of claim 1 wherein an anterior, trans inferior endplate trajectory is accomplished for treating intervertebral disc abnormality.

9. The method of claim 1 wherein an inferior endplate with an anterior, trans inferior endplate trajectory is accomplished for treating intervertebral disc abnormality.

10. The method of claim 1 to provide an access route for placing, congealing, polymerizing and securing intervertebral disc space implants for treating intervertebral disc abnormality.
